(19) United States
(12) Patent Application Publication
(10) Pub. No.: US 2008/0208123 A1
(43) Pub. Date: Aug. 28, 2008

(54) ADMINISTERING DEVICE WITH COMPULSORY PRIMING

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/941,511

(22) Filed: Nov. 16, 2007

Related U.S. Application Data
(63) Continuation of application No. PCT/CH2006/000236, filed on Apr. 28, 2006.

(30) Foreign Application Priority Data
May 17, 2005 (DE) 10 2005 022 532.2

(51) Int. Cl. A61M 5/36

(52) U.S. Cl. 604/123

(57) ABSTRACT

A device for administering a product after executing a priming process, the device including a housing for accommodating an ampoule containing the product, a mechanism for moving a plug or piston in the ampoule, and a lock arrangement operably coupled to the mechanism which prevents an administration of the product without a previous execution of the priming process, wherein, upon or after executing the priming process, the lock arrangement is released and the mechanism is operable for administering the product.

### CROSS-REFERENCE TO RELATED APPLICATIONS

This application is a continuation of International Application No. PCT/CH2006/000236 filed on Apr. 28, 2006, which claims priority to German Application No. DE 10 2005 022 532.2 filed on May 17, 2005, the contents of both of which are incorporated in their entirety herein by reference.

### BACKGROUND

The present invention relates to devices for administering, delivering, injecting, infusing or dispensing a substance, and to methods of making and using such devices. More particularly, it relates to an administering device for administering a product after a priming procedure has been performed, wherein the device comprises a casing for accommodating the product and/or an ampoule containing the product and a mechanism for advancing a stopper in the ampoule to administer the product. The product may be intended to be injected into an organism, in particular a human being.

Various administering devices, some of which are known as injection pens, are known in the prior art and may be used to administer medicinal substances, for example insulin or the like, to patients. In such administering devices, it is necessary to perform a priming procedure before an administration, i.e. to remove air and any impurities from a cannula forming part of the device, by rinsing with the product itself. It is not unusual for the priming procedure to be forgotten, particularly in stressful situations, for instance when a diabetic is close to a state of shock in which an insulin injection is urgently required.

### SUMMARY

It is an object of the present invention to provide an administering device in which it is almost impossible to omit a priming procedure.

In accordance with the present invention, an administering device is provided which exhibits the advantages that on the one hand, the priming procedure cannot be forgotten before an administering procedure, and on the other hand, the priming procedure can be performed as a compulsory procedure and activated with little effort.

In one embodiment, the present invention comprises a device for administering a product after executing a priming process, the device comprising a housing for accommodating an ampoule containing the product, a mechanism for moving a plug or piston in the ampoule, and a lock arrangement operably coupled to the mechanism which prevents an administration of the product without a previous execution of the priming process, wherein, upon or after executing the priming process, the lock arrangement is released and the mechanism is able to administer the product.

In some embodiments, the invention is used in disposable administering devices, but it can also be used in reusable administering devices.

In one embodiment, the present invention comprises an administering device for administering a product after a priming procedure has been performed, comprising a casing accommodating an ampoule containing the product, a mechanism for advancing a stopper in the ampoule, and a lock operably coupled to the mechanism, said lock preventing an administering of the product when the priming procedure is not first performed; wherein performing the priming procedure unlocks the lock.

In one embodiment, the present invention comprises a method of administering a substance, the method involving an administering device for administering a product after a priming procedure has been performed, the device comprising a casing for accommodating an ampoule containing the product and a mechanism for advancing a stopper in the ampoule, wherein a blocking arrangement is associated with the mechanism for advancing and obstructing the mechanism if the priming procedure is not performed first, said method comprising the steps of performing the priming procedure whereby the blocking arrangement is released, then administering the product.

The advantages which can be achieved in accordance with the present invention are based on the fact that a blocking arrangement, lock or lock mechanism is assigned or operably coupled to the mechanism for advancing the stopper within the ampoule, and obstructs an administration of the product without the priming procedure having been performed beforehand, wherein performing the priming procedure unlocks the blocking arrangement and releases the mechanism for administering the product, enabling it to be actuated.

In one preferred embodiment, the device in accordance with the present invention comprises a priming button, via which a blocking arrangement or lock can be unlocked, wherein activating the priming button moves a blocking unit out of a blocking position to release an administering mechanism, e.g., an administering button of the mechanism, for administering the product. In one preferred embodiment, the administering device in accordance with the present invention comprises two activating buttons, one for the priming procedure which is unavoidably to be performed and a second which is only to be activated subsequently to administer the product. This simple manageability ensures that even in stressful situations, a patient is capable of performing the necessary procedures.

In accordance with another preferred embodiment, the blocking arrangement in accordance with the present invention comprises a switching member which, in a blocking position, prevents an administering button from being activated, wherein the switching member can be deflected out of the blocking position, such that the administering button can be activated. In this exemplary variant, too, it is possible to ensure, in stressful situations, both simple manageability and the essential order of the procedures to be performed.

It should be noted that the invention can be used in a “throw-away” or disposable administering device, since in such a case, the blocking arrangement and the administering mechanism can be embodied to be relatively simple, since they only have to be configured in one direction, i.e. the direction in which the stopper is to be advanced.

In some preferred embodiments, a switching member spring serves the switching member to deflect the switching member out of the blocking position. By activating the priming button, the patient can ensure that the switching member spring is activated and deflects the switching member out of the blocking position. Simple activation is also possible here, and the patient need only then worry about pushing or activating the administering button.

In accordance with another preferred embodiment, the aforementioned blocking unit can block the switching member in the blocking position.

In some preferred embodiments, the blocking arrangement or lock includes a priming rod which acts on the stopper when the priming button is activated. While the priming procedure is thus performed, this also releases the mechanism for advancing the stopper to administer the product. In some preferred embodiments, the mechanism for advancing comprises an administering rod which acts on the stopper as a result of an administering button being activated.

In some preferred embodiments, a spring, e.g., a compressed spring, is also assigned or operably coupled to the administering rod and advances the administering rod, which then advances the stopper within the ampoule to administer the product.

In some preferred embodiments, the administering rod and the priming rod are arranged coaxially with respect to each other, or in each other, which can simplify the design of the present invention even further. The rods can, of course, also be respectively arranged at an angle to each other.

One exemplary preferred embodiment in accordance with the present invention is explained below in more detail by referring to the enclosed figures, wherein additional features, objects and advantages in accordance with the present invention are disclosed. In the drawings, identical or at least functionally identical components are cited using the same reference numerals.

### BRIEF DESCRIPTION OF THE DRAWINGS

FIG. 1 is an axial longitudinal section through a preferred embodiment of a device in accordance with the present invention; and

FIG. 2 is an axial longitudinal section through the embodiment of FIG. 1, rotated by 90°.

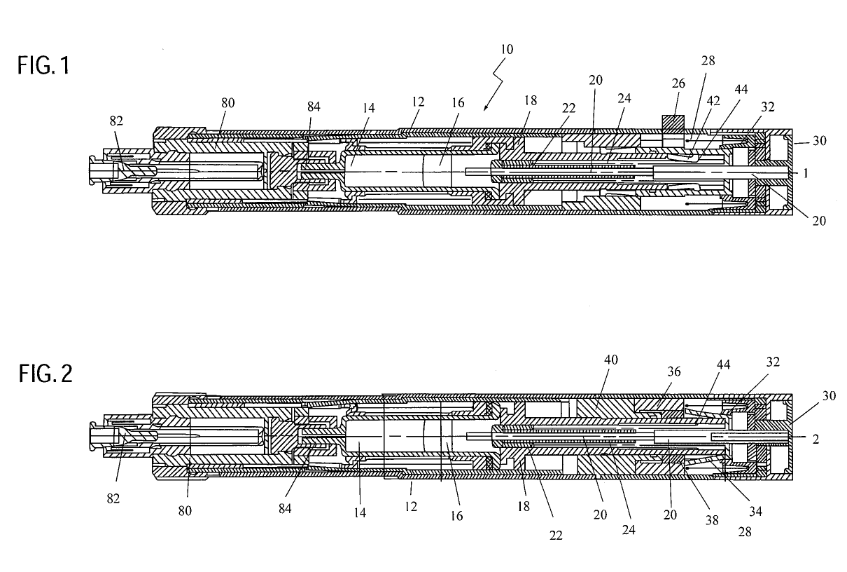

### DETAILED DESCRIPTION

In one embodiment, the present invention comprises a device 10 for administering a product comprising a casing 12, into which an ampoule 14 containing the product to be administered is inserted. A stopper 16 is arranged in the ampoule 14 and is shifted in the axial direction towards a cannula 82 at the front end of the device 10 to administer the product and perform a priming procedure.

The ampoule 14 is attached or flanged onto the administering mechanism via an ampoule bearing 18. If the device in accordance with the present invention is a device which is to be used once, the ampoule can already be flanged on at the manufacturer's. Otherwise, this procedure may be performed by the patient whenever a new, full ampoule 14 is to be inserted into the administering device 10.

A cannula mounting 80 is provided at the front end of the device 10 and holds the cannula, or needle, 82. The cannula mounting can be removed to the mid-cannula, and the cannula can thus be attached to a cannula attachment 84 on the ampoule 14 without direct contact and therefore sterilely to administer the product or perform a priming procedure.

A priming button 30 is provided at the end of the device 10 opposite the cannula end. The priming button 30 acts on a priming rod 20 which can extend in the ampoule up to the stopper 16. When pushed or activated, the priming button 30 also acts on a blocking member release 32 which is also shifted axially towards the cannula end of the device 10 when the priming button 30 is activated. Pushing or activating the priming button 30 thus moves the blocking member release 32 forward, thus moving in blocking arms 34 which are spread or fanned out in an initial state. FIG. 2 shows the blocking arms 34 in their functional position. Activating the priming button 30 thus not only moves the priming rod 20 forward, but also deflects the blocking arms 34 radially inwardly, which disengages the blocking arms 34, which are supported on a switching member 36, from the switching member 36. This releases the switching member 36, and a switching member spring 28 draws the switching member toward the priming button 30, as can be seen from FIG. 2.

An administering button 26 cannot be pushed or activated as long as the switching member 36 is in its original position, and as soon as the switching member 26 has been pulled out of its blocking position, the blocking limbs 38—which are formed onto the administering button 36—can be moved perpendicular to the axis of the device 10. This releases blocking fingers 42 which have been holding an administering rod 22 in an initial position, wherein in said position, an administering spring 24 is held in a biased position. As soon as the blocking fingers 42 are no longer gripping or lodged behind a collar 44 of the administering rod 22, the administering rod is consequently advanced forwards, toward the cannula region of the device 10, and pushes the stopper 16 forward to perform an administering procedure.

The mechanism for administering is held within a guiding block 40 in the casing 12, wherein the guiding block 40 is held within a casing which may also comprise multiple parts. The ampoule bearing 18 can also be held on the casing, such that the casing—together with the guiding block 40 and the ampoule bearing 18—forms a static base construction or unit within which the administering mechanism and the blocking arrangement are held and/or guided.

With regard to the described embodiment, it should be noted that the catching arms 34 can be used directly for the purpose of performing a blocking function in relation to the blocking limbs 38 and/or blocking fingers 42. For this purpose, the blocking arms 34 would be in engagement with a collar (not shown) on the blocking limbs 38 in their blocking state (as shown), and activating the priming button 30 and advancing the blocking member release 32 would disengage the blocking arms 34 from the collars or abutments (not shown) on the blocking limbs. In this case, a blocking function could then be performed without the switching member 26, and cancelled by performing a priming procedure. This would also be possible if only the switching member 36 were provided, wherein activating the priming button 30 could move the switching member from its original position as shown by deflection without using the blocking arms 34 to release the administering button 26.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An administering device for administering a product after a priming procedure has been performed, comprising:
a casing accommodating an ampoule containing the product;
a mechanism for advancing a stopper in the ampoule; and
a lock operably coupled to the mechanism, said lock preventing an administering of the product when the priming procedure is not first performed; wherein
performing the priming procedure unlocks the lock.

2. The administering device according to claim 1, further comprising a priming button, and the lock is unlocked via the priming button.

3. The administering device according to claim 2, further comprising an administering button, wherein the lock comprises a switching member having a locked position wherein the administering button can not be activated, and wherein the switching member can be moved from the locked position so the administering button can be activated.

4. The administering device according to claim 3, further comprising a switching member spring that urges the switching member out of the locked position.

5. The administering device according to claim 4, wherein the lock comprises a priming rod which acts on the stopper when the priming button is activated to perform the priming procedure.

6. The administering device according to claim 5, wherein the mechanism for advancing the stopper comprises an administering rod which acts on the stopper as a result of the administering button being activated.

7. The administering device according to claim 6, further comprising an administering spring which acts on the administering rod and advances it to administer the product.

8. The administering device according to claim 7, wherein the priming rod and the administering rod are coaxial.

9. An administering device for administering a product after a priming procedure has been performed, comprising:
a casing for accommodating an ampoule containing the product and a mechanism for advancing a stopper in the ampoule;
a blocking arrangement associated with the mechanism, said blocking mechanism obstructing administering of the product without first performing the priming procedure; wherein
performing the priming procedure unlocks the blocking arrangement and releases the mechanism for administering the product, enabling it to be activated.

10. The administering device according to claim 9, wherein the blocking arrangement can be unlocked via a priming button, wherein activating the priming button moves a blocking unit out of a blocking position to release the mechanism for administering the product, said mechanism comprising an administering button.

11. The administering device according to claim 10, wherein the blocking arrangement comprises a switching member which, in a blocking position prevents an administering button from being activated, wherein the switching member can be deflected out of the blocking position such that an administering button associated with the mechanism for administering can be activated.

12. The administering device according to claim 11, further comprising a switching member spring that urges the switching member out of the blocking position.

13. The administering device according to claim 11, wherein the blocking unit blocks the switching member in the blocking position.

14. The administering device according to claim 10, wherein the blocking arrangement comprises a priming rod which acts on the stopper when the priming button is activated to perform the priming procedure, and wherein the mechanism for advancing the stopper is released to administer the product.

15. The administering device according to claim 14, wherein the mechanism for advancing the stopper comprises an administering rod which acts on the stopper as a result of the administering button being activated.

16. The administering device according to claim 15, further comprising an administering spring acting on the administering rod to administer the product.

17. The administering device according to claim 16, wherein the priming rod and the administering rod are one of arranged coaxially with respect to each other or one inside the other.

18. A method of administering a substance, the method involving an administering device for administering a product after a priming procedure has been performed, the device comprising a casing for accommodating an ampoule containing the product and a mechanism for advancing a stopper in the ampoule, wherein a blocking arrangement is associated with the mechanism for advancing and obstructing the mechanism if the priming procedure is not performed first, said method comprising the steps of performing the priming procedure whereby the blocking arrangement is released, then administering the product.
